(19) United States
(12) Patent Application Publication
(10) Pub. No.: US 2025/0170027 A1
(43) Pub. Date: May 29, 2025

(54) COLLAGEN CROSS-LINKER AND USES THEREOF

(71) Applicant: The Curators of the University of Missouri, Columbia, MO (US)

(72) Inventors: Zhonghua PENG, Overland Park, KS (US); Yong WANG, Overland Park, KS (US); Viviane HASS, Overland Park, KS (US); Yong LI, Kansas City, MO (US); Rong WANG, Leawood, KS (US)

(21) Appl. No.: 18/724,765

(22) PCT Filed: Feb. 3, 2023
(86) PCT No.: PCT/US23/12305

§ 371 Date: Jun. 27, 2024

Related U.S. Application Data
(60) Provisional application No. 63/306,748, filed on Feb. 4, 2022.

(51) Int. Cl. A61K 6/20; A61K 6/60

(52) U.S. Cl. CPC A61K 6/20; A61K 6/60

(57) ABSTRACT

Crosslinking agents, TRI-1, TRI-2, and TRI-3, and/or a collagen crosslinking mixture comprising two or more of TRI-1, TRI-2 and TRI-3, are presented, as are the syntheses thereof. The collagen crosslinking effects of the mixture of TRI-1, TRI-2, and TRI-3 are significantly enhanced over any of the molecules taken individually. The mixture drastically improves collagen biostability, showing enhanced performance compared to commercially available collagen treatments, and acts quickly, requiring no more than 60 seconds to show inhibition of collagenases. The mixture is also nontoxic and has a slight beige color, which is aesthetically ideal for any treatment that may be visible to others. The collagen crosslinker mixture is furthermore shown to be mixable with commercial dental adhesive without compromising the desired properties of the crosslinkers or the adhesive.

### CROSS-REFERENCE TO RELATED APPLICATIONS

The present application is the US national stage under 35 U.S.C. § 371 of International Application No. PCT/US2023/012305 which was filed on Feb. 3, 2023, and which claims the priority of U.S. provisional patent application Ser. No. 63/306,748 filed Feb. 4, 2022.

### STATEMENT REGARDING FEDERALLY SPONSORED RESEARCH OR DEVELOPMENT

This invention was made with government support under Grant/Contract/Award 0053665 awarded by National Institutes of Health (NIH). The government has certain rights in the invention.

### FIELD

The present teachings relate to biocompatible mixtures of novel organic cross-linkers (crosslinking agents) for stabilizing collagen, as well as methods of preparing and using same.

### BACKGROUND

The statements in this section merely provide background information related to the present disclosure and may not constitute prior art.

Collagen is the primary structural protein found in connective tissues throughout the human body, including skin, cartilage, ligaments, tendons, bones, and teeth. In part due to the breadth of its applications in the body, collagen mixtures can be significantly varied, such that there are twenty-eight known types of collagen in the body. Nevertheless, all known types are known to include at least one triple helix of three polypeptide chains. The most common type in the human body, called Type I collagen, assembles into fibers that form the structural and mechanical scaffold of bone, skin, tendons, cornea, blood vessel walls and other connective tissues. Type I collagen is involved in the development, formation, and homeostasis of these tissues, providing structure and strength as well as playing a role in tissue repair.

Biosynthesis of collagen occurs naturally, although the rate at which collagen is biosynthesized slows as we age. Degradation of collagen also occurs naturally, often modulated by enzymes called collagenases. It is tied to negative outcomes such as the sagging and wrinkling of epidermal tissues. The restoration and support of naturally degraded collagen is thus a growing focus of the so-called anti-aging industry. Therapies directed toward restoration and support of collagen, particularly when topical, are ideally colorless and non-toxic, both to maintain aesthetic appeal and to ensure that the user is not harmed by the therapy.

Many therapies that seek to restore and safeguard collagen work slowly over time. These include behavioral therapies, such as the avoidance of tobacco, excess sugar, and excess sunlight, as well as supplement-based therapies such as the measured intake of hyaluronic acid. However, collagen loss and degradation can also be pathological or trauma-induced, and such acute collagen degradation can potentially hinder healing and interfere with medical interventions. Some collagen treatments are applied directly to bodily collagen and attempt to restore and safeguard that collagen by strengthening collagen fibrils and enhancing their mutual entanglement.

Such bolstering of the mutual entanglement of collagen fibrils may be via crosslinking. “Crosslinking” is a broad term in the chemical and biological sciences, but it encompasses both physical means (wherein the crosslinker enhances weak attractive interactions between fibrils) and chemical means (wherein the crosslinker directly covalently bonds with multiple fibrils to create ‘bridges’ that link those fibrils). While crosslinking can be a powerful means of strengthening polymers and proteins such as collagen, it does require care, as the introduction of exogenous chemicals into the body can raise concerns about toxicity and aesthetic. Furthermore, many known crosslinkers may exhibit some capacity to improve the strength and/or entanglement of collagen fibrils, but are themselves vulnerable to common chemical reactions that degrade them. Such crosslinkers are considered to have reduced ‘biostability’ because they are less capable of retaining stability once applied to bodily tissues. Thus, there also exists a need for collagen crosslinkers with high biostability that can be locally applied to a site of sudden and/or acute collagen degradation.

### SUMMARY OF THE INVENTION

In various embodiments, the present disclosure provides three novel collagen crosslinking agents referred to herein as TRI-1, TRI-2, and TRI-3. The present disclosure additionally provides a mixture comprising two or more of the three crosslinking agents TRI-1, TRI-2, and TRI-3. Although each of TRI-1, TRI-2, and TRI-3 is novel and independently provide the advantages described herein, a mixture comprising two or more, e.g., a combination of all three, provides greater crosslinking performance (e.g., inhibition of collagenases) than any of the individual crosslinking agents TRI-1, TRI-2 and/or TRI-3 in isolation. Particularly, a mixture of two or more of the crosslinking agents TRI-1, TRI-2, and TRI-3, when applied to collagen, drastically improves collagen stability by improving collagen fibril crosslinking. The extent of collagen stabilization provided by the crosslinking agents and/or mixture thereof described herein is far enhanced compared to commercially available collagen treatments, as described in detail below. The inhibition of collagenases can be directly inferred from the extent of collagen degradation upon exposure to collagenases. Additionally, in various embodiments, the herein disclosed crosslinkers and/or the mixture thereof requires no more than 60 seconds from initial exposure to show strong inhibition of collagenases, thereby protecting collagen far more rapidly than is seen with commercially available collagen treatments.

In various embodiments, the crosslinkers and/or the mixture thereof is also colorless, which is aesthetically ideal for any treatment that may be visible to the human eye, and it is nontoxic. In various embodiments, the collagen crosslinkers and/or the mixture thereof of the present disclosure actually show great biocompatibility and even improves dentin cell proliferation. The collagen crosslinkers and/or the mixture thereof of the present disclosure are furthermore shown to be mixable with commercial dental adhesives without compromising the desired colorlessness and fast-acting, non-toxic collagen stabilizing properties of the crosslinker mixture or the polymerization properties of the dental adhesive.

Also disclosed herein is a method for synthesizing the crosslinking mixture of the present disclosure.

### BRIEF DESCRIPTION OF THE DRAWINGS

FIGS. 1A and 1B exemplarily show simplified synthetic routes for each of three components of a biocompatible collagen crosslinker mixture, in accordance with various embodiments of the present disclosure. FIG. 1A shows a fully generalized synthetic route, wherein the reaction of a catecholamine and a tricarbonyl benzene produces molecules of the generalized forms of TRI-1a, TRI-2a, and TRI-3a, depending on the nature of R substituents, which are independently selected. FIG. 1B provides a more specific example of the generalized synthesis modeled in FIG. 1A, where the reaction of trimesoyl chloride and dopamine produces TRI-1, TRI-2, and TRI-3.

FIGS. 2-9 exemplarily illustrate experimental protocols provided in the “examples” described below.

FIGS. 2A and 2B are exemplary bar graphs showing the biostability of dentin collagen against collagenase given various crosslinking agents. FIG. 2A shows the percent weight loss of dentin collagen associated with each crosslinker; FIG. 2B shows the amount of hydroxyproline released from dentin collagen associated with each crosslinker.

FIG. 3 is a bar graph comparing the extents of collagenase activity on dentin collagen after given periods of continuous exposure to known crosslinking agents versus exposure to the novel crosslinking mixture of the present disclosure.

FIGS. 4A and 4B compare how the crosslinking mixture of the present disclosure and various known crosslinking agents in terms of their modulation of matrix metalloproteinase activity on dentin collagen. FIG. 4A shows fluorescence microscopy images of dentin samples alternately exposed to the herein disclosed crosslinking mixture and the other known crosslinking agents for 60 seconds (top images) and 1 hour (bottom images); FIG. 4B is a bar graph depicting matrix metalloproteinase activity after given periods of exposure of dentin collagen samples of FIG. 4A to the herein disclosed crosslinking mixture and various crosslinking agents.

FIG. 5 is a bar graph showing relative intensities of absorbance spectroscopy of L929 cells treated with the crosslinking agents shown in FIGS. 4A and 4B to compare the capacities of the herein disclosed crosslinking mixture as well as various known crosslinking agents for stabilizing dentin collagen and thereby improving cell proliferation across given concentrations and periods of exposure.

FIGS. 6A and 6B show bar graphs depicting counts of live L929 cells and dead L929 cells, respectively, after exposing cells to dentin collagens treated with the herein disclosed crosslinking mixture or various known crosslinkers for an extended period of one day or three days.

FIGS. 7A and 7B show fluorescence microscopy images of live (top row) and dead (bottom row) L929 cells after exposure for 1 and 3 days to substances released from dentin collagens treated with the herein disclosed crosslinker mixture as well as various known crosslinkers.

FIGS. 8A and 8B are comparative photographs of dentin films before (top images) and after collagenase digestion (bottom images). The dentin films were treated with a commercial dental adhesive alone, the herein disclosed crosslinker mixture alone, or the dental adhesive with the herein disclosed crosslinker mixture in various weight percentages.

FIG. 9 is a bar graph comparing the degrees of conversion of a commercial dental adhesive mixed with fixed concentrations of either the herein disclosed crosslinking mixture or a known crosslinking agent, GSE.

Corresponding reference numerals will be used throughout the several figures of the drawings.

### DETAILED DESCRIPTION

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what we presently believe is the best mode of carrying out the claimed invention. Additionally, it is to be understood that the claimed invention is not limited in its applications to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

The terms “crosslinker” and “crosslinking agent” as used herein refer to any substance that, by means of covalent bond formation, ionic bond formation, hydrogen bond formation, hydrophobic interactions or any furthering of physical entanglement, strengthens the attractive interactions between strands of a polymer or protein.

The term “biocompatible” as used herein describes materials and/or therapies that are effective as intended while not having toxic, injurious, or otherwise known significant deleterious effects on biological systems.

The term “small molecule” as used herein refers to organic chemical compounds whose molecular weight is equal to or less than 1,000 Daltons. Concordantly, the term “large molecule” as used herein refers to organic chemical compounds whose molecular weight is greater than 1,000 Daltons.

The term “biomolecule” as used herein refers to biological material such as proteins, peptides, carbohydrates, and polymers naturally formed from biological monomers.

The term “collagen” as used herein refers to any of the known types of collagen found in the bones, teeth, skin, and other tissues of animal species, without prejudice to the effects on collagen mixture and morphology from a surrounding chemical or biological matrix.

The term “dentin” as used herein refers to the major component of teeth found just below the enamel, the organic mixture of which is 90% type I collagen.

The term “collagenase” as used herein refers to any enzyme that denatures collagen. Herein, it most commonly refers to collagenase type I, which is a protease that cleaves a glycine-amino acid bond found in high frequency in collagen.

The term “MMP” as used herein refers to matrix metalloproteinase, which is a group of enzymes capable of degrading multiple extracellular matrix proteins, bioactive molecules, and, crucially, collagen.

The term “HYP” as used herein refers to hydroxyproline, which is a major component of collagen in the human body.

The term “MTS” as used herein refers to (3-(4,5-dimethylthiazol-2-yl)-5-(3-carboxymethoxyphenyl)-2-(4-sulfophenyl)-2H-tetrazolium, which is a reagent commonly used in colorimetry assays on cell cultures.

The term “EDTA” as used herein refers to ethylenediaminetetraacetic acid, a well-known chelating agent and selective inhibitor against various enzymes.

The term “acetonitrile” as used herein is a compound also known as methyl cyanide with the formula CH₃CN.

The term “EtOH” as used herein refers to ethanol.

The term “THF” as used herein refers to tetrahydrofuran.

The term “TEA” as used herein refers to triethylamine, not to be confused with triethanolamine.

The term “EGCG” as used herein refers to epigallocatechin gallate, a known crosslinker.

The term “QC” as used herein refers to quercetin, a known crosslinker.

The term “GEN” as used herein refers to genipin, a known crosslinker.

The term “EDC/NHS” as used herein refers to 3-dimethylaminopropyl-carbodiimide hydrochloride/N-hyroxy-succinimide, a known crosslinker.

The term “GSE” as used herein refers to grape seed extract, which is known to have a protective effect on collagen.

The terms “CT” and “CONTROL” as used herein refer to a control test for a particular experiment or study.

The term “GA” as used herein refers to glutaraldehyde.

The term “NCTC” as used herein refers to the National Collection of Type Cultures, a repository that collects, stores, and distributes biological culture reference substances.

The term “ATCC” as used herein refers to the American Type Culture Collection, a repository that collects, stores, and distributes biological culture reference substances.

The term “FBS” as used herein refers to fetal bovine serum, a growth supplement for cell culture media.

The term “MALDI” as used herein refers to matrix-assisted laser desorption/ionization, a technique in mass spectrometry that ionizes samples using pulsed laser irradiation and is typically applied to large molecules and biomolecules.

The term “ESI” as used herein refers to electrospray ionization, a technique in mass spectrometry that ionizes samples by applying high voltage to the sample.

The term “TOF” as used herein refers to “time-of-flight,” a type of mass spectrometry in which the mass/charge ratios of ions in a sample are determined by the time taken for a given ion in a fixed electric field to reach a detector at a known distance from the ionization point.

The term “CLSM” as used herein refers to confocal laser scanning microscopy, which is a microscopy technique that finely controls optical resolution and depth of field.

The term “ANOVA” as used herein refers to “analysis of variance,” a standard group of statistical models that are often used to determine whether the mean values of two or more populations are statistically equal.

The term “catecholamine” as used herein refers to a class of compounds defined by a benzene ring featuring two adjacent alcohol groups as well as an alkyl amine. This includes a bare catecholamine or a catecholamine featuring further substitutions. Compounds of the form:

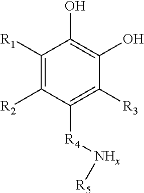

where R₁-R₄ are each independently selected from hydrogen, halides, and alkyl groups, including linear alkyl groups of various chain length, are specifically included. R₅ is a hydrogen or an alkyl group. Hₓ can represent one or two hydrogens, and the amine can therefore be a primary, secondary, tertiary, or quaternary amine. In quaternary amine form, any suitable anion can counter the amine's charge. As used herein, the term catecholamine is thus not restricted to the three well-known neurotransmitters, dopamine, epinephrine, and norepinephrine, although these are included in the scope of the term.

The term “dopamine” as used herein is a contraction of the molecule 3,4-dihydroxyphenethylamine. The term “dopamine hydrochloride” refers to the hydrochloride salt of dopamine, the chemical formula of which is C₈H₁₂NO₂Cl.

The term “tricarbonyl benzene” as used herein refers to a molecule featuring a benzene that comprises three carbonyl substituents. Molecules of the form:

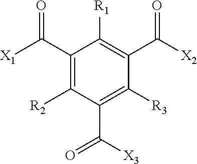

where R₁-R₃ are each independently selected from hydrogen, halides, and alkyl groups, and where X₁-X₃ are each independently selected from hydrogen, oxygen, nitrogen, halides, alkyl oxides, and alkyl amines, are specifically included. The tricarbonyl benzenes of the present disclosure are preferentially selected such that the primary site of reactivity for nucleophilic reagents will be the carbonyl centers.

The term “trimesoyl chloride” as used herein refers to the molecule also known as 1,3,5-benzenetricarbonyl trichloride.

The term “TRI” as used herein is a contraction of the term “tri-amide,” and refers broadly to the collagen crosslinker mixture of the present disclosure. The specific terms “TRI-1,” “TRI-2,” and “TRI-3” as used herein refer to the molecular components of this disclosure's collagen crosslinker mixture, and can be identified by the fact that they feature, respectively, three amide groups, two amide groups and a carboxylic acid, and two amide groups and an ester.

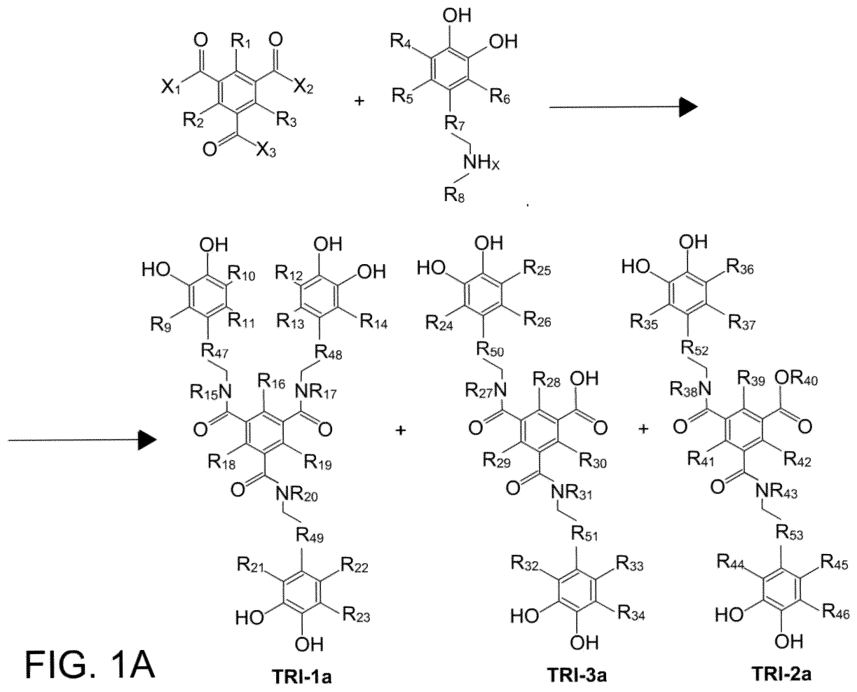

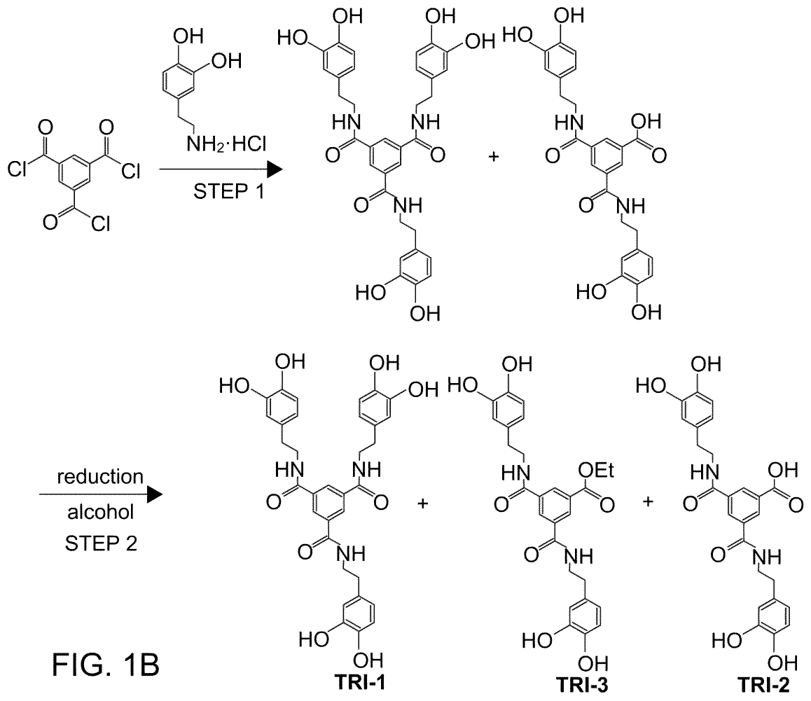

Referring now to FIG. 1A-1B, in various embodiments, the present disclosure provides three newly discovered generalized forms of small molecule collagen crosslinkers (TRI-1a, TRI-2a, and TRI-3a), three specific variations of these crosslinkers (TRI-1, TRI-2, and TRI-3), as well as a mixture comprising two or more of the TRI-1, TRI-2, and TRI-3. When applied to collagen, these three crosslinkers, and/or the combination of two or more in a single mixture, stabilize collagen fibrils to help protect against degradation of collagen from sources such as collagenases. While each of TRI-1, TRI-2, and TRI-3 is novel, this mixture of two or more, e.g., all the three, is also synergistic, providing significantly enhanced collagen protection beyond that of any of the individual components in isolation. The structures of each of TRI-1, TRI-2, and TRI-3 comprise catechol-like end groups that are theorized to aid in their ability to form attractions to collagen. Furthermore, these structures are mostly free of the reactive ester groups found in the prior art. This comparative lack of reactivity is thought to contribute to biostability provided by TRI-1, TRI-2, and TRI-3 crosslinkers individually and/or combination. As disclosed below, in various embodiments, it is possible to produce all three of these molecules in a desired stable, highly synergistic combination in a single synthetic sequence.

As exemplarily illustrated in FIG. 1A, in various embodiments, a collagen crosslinker mixture comprising generalized molecules TRI-1a, TRI-2a, and TRI-3a can be synthesized via simple reactions of inexpensive commercially available compounds: a tricarbonyl benzene compound and a catecholamine. FIG. 1A provides a highly generalized scheme for synthesis of the crosslinking mixture of the present disclosure, where “R” and “X” substituents denote sites at which ordinary, minor variations can be envisioned by those of ordinary skill in the art without significantly altering the mixture's properties and deviating from the scope of this disclosure. Further alterations to the disclosed synthesis and reagents may also be envisioned by those of ordinary skill without significantly altering the mixture's properties, and these are also considered to be within the scope of this disclosure.

FIG. 1B provides a more specific synthetic scheme as performed in various embodiments. A full exemplary synthesis is provided later in the section “Synthesis A.” Particularly, FIG. 1B exemplarily shows trimesoyl chloride and dopamine hydrochloride undergoing addition-elimination reaction in step 1, resulting in the fully-substituted compound TRI-1, which contains three amide groups, as well as the partially-substituted TRI-2, which features two amide groups and a carboxylic acid group. This mixture comprising TRI-1 and TRI-2 is collected and washed, then undergoes further reaction with a reducing agent and an alcohol to form the mixture of TRI-1, TRI-2, and the molecule TRI-3, which features two amide groups and an ethyl ester. Thus, the present disclosure provides the synergistic mixture comprised of novel collagen crosslinkers TRI-1, TRI-2 and TRI-3 that can be prepared in two steps. Careful control of the synthetic process can influence the ratios of TRI-1, TRI-2 and TRI-3 within the mixture, thereby affecting the efficacy of the mixture for stabilizing collagen from natural sources such as dentin, skin, bone, etc.

In various embodiments the structures TRI-1 (C₃₃H₃₃N₃O₉), TRI-2 (C₂₅H₂₄N₂O₈), and TRI-3 (C₂₇H₂₈N₂O₈), can be intermixed at various ratios to provide various benefits described in herein, mainly collagen biostability. The weighted percentage range can be from near zero to 100 for each, and more precisely as a:b:c=x:y:(100−x−y), where 0≤x<90 and 10<y≤100. In various embodiments, the percentage ratio a:b:c can be approximately 50:25:25.

In various embodiments, the reactions of steps 1 and 2 in FIG. 1B can be better controlled by placing the reaction vessels in NaCl/ice baths under flowing N₂. In various embodiments, the solvent used for step 1 of FIG. 1B is a polar aprotic solvent such as acetonitrile, while the solvent used for step 2 of FIG. 1B can be THF. In various embodiments, the reaction of step 1 can be enhanced by the addition of triethylamine. In various embodiments, the reaction of step 2 is a reduction performed with LiAlH₄ and worked up with hydrochloric acid and ethanol. However, although exemplary reagents are listed for the general reactions shown here, a practitioner of ordinary skill in the art will recognize acceptable substitutions and alterations, all of which are within the scope of present disclosure.

For example, in various exemplary embodiments, a polar aprotic solvent other than acetonitrile can be used, including but not limited to dichloromethane, tetrahydrofuran, ethyl acetate, dimethyl formamide, dimethyl sulfoxide, and acetone. Similarly, a polar aprotic solvent other than THE can be used during the addition of reducing agent.

In various embodiments, an alkaline compound other than triethylamine can be used, including but not limited to pyridine, N,N-diisopropylethylamine, and potassium carbonate.

The NaCl/ice baths are, in various embodiments, prepared in a ratio of 99 grams of NaCl to 300 grams of ice. The temperature of the NaCl/ice baths is in various embodiments −21.3° C., but may be anywhere in the range of −21.3° C. to 0° C. Instead of being a mixture of NaCl and ice, the NaCl/ice baths can be any mixture that provides a low-temperature environment.

In various embodiments, reactions can be performed under any inert environment instead of N₂, such as Ar.

In various embodiments, a reducing agent other than LiAlH₄ can be used, for example a weaker reducing agent, such as sodium borohydride.

Experiments show that application of any one of the crosslinkers TRI-1, TRI-2 and TRI-3, or the collagen crosslinker mixture comprising two or more of the TRI-1, TRI-2 and TRI-3, e.g., mixture 10 comprising all three crosslinkers TRI-1, TRI-2 and TRI-3, to mammalian collagen improves the biostability of that collagen. Numerous studies assessing the individual crosslinkers TRI-1, TRI-2 and TRI-3 and crosslinking mixture, e.g., crosslinking mixture 10 demonstrate positive effects particularly on dentin collagen. The following specific examples provide detailed information on a synthetic protocol for a collagen crosslinker mixture found beneficial to dentin collagen as well as the particular studies conducted on the interaction between the mixture 10 and the dentin collagen.

### SPECIFIC EXAMPLES

### Synthesis A

In one exemplary embodiment, acetonitrile and TEA were dried with freshly activated 3 Å and 4 Å molecular sieves, respectively, for over 24 h before use. Dopamine hydrochloride (3.3 eq.; 2.0381 g, 10.75 mmol) was dissolved in 40 mL of dry CH₃CN under N₂. To the stirred solution, dry TEA (12 eq.; 3.8857 g, 5.35 mL, 38.4 mmol) was added. The reaction vessel was moved to an ice/NaCl bath (mixture of 99 g of NaCl and 300 g of ice, −21.3° C.) and kept stirring for additional 15 min before a solution of trimesoyl chloride (1 eq.; 0.8535 g, 3.22 mmol) in 20 mL of dry CH₃CN was added dropwise over the course of ˜30 min. The reaction mixture was kept stirring under N₂ in the gradually melting ice/NaCl bath for over 44 h. The reaction mixture was vacuum-filtered and the filtrate was rotary evaporated under vacuum to remove the acetonitrile solvent. The solid from vacuum filtration and the solid recovered from evaporation were combined and washed with 50 mL of 2 M HCl aqueous solution with the assistance of sonication, stirring, and physical grinding (pH<2). After vacuum filtration, the solid was washed thoroughly with deionized (DI) water. The solid (1.2656 g) was dried at 30° C. under vacuum for over 48 h. A MALDI TOF mass spectrometer, calibrated with CsI₃, was used to analyze the sample. The sample (4.96 mg) was dissolved in 200 μL of CHCl₃ and the solution was mixed with commercially-acquired MALDI matrix 2,5-dihydroxybenzoic acid solution (1:1) and spotted onto the MALDI plate. A major peak at m/z 616.2272, corresponding to [M+H]⁺ (m/z 616.2290) of TRI-1 (C₃₃H₃₃N₃O₉, monoisotopic mass: 615.2217, theoretical [M+H]⁺: 616.2290) and a minor peak at m/z 481.1679, corresponding to [M+H]⁺ (m/z 481.1605) of TRI-2 (C₂₅H₂₄N₂O₈, monoisotopic mass: 480.1533, theoretical [M+H]⁺: 481.1605) were observed. The molar ratio of TRI-1:TRI-2 is calculated to be 88.15:11.85.

The above product mixture (307.8 mg) was dispersed in THF (15 mL) and the mixture was stirred under nitrogen atmosphere in an ice/NaCl bath (mixture of 99 g of NaCl and 300 g of ice, −21.3° C.) for ˜15 min. To the mixture was added 3 mL of 2.4 M LiAlH₄ in THE dropwise. The ice bath was removed, and the reaction mixture was heated to reflux and kept refluxing for 2 h. After cooled to room temperature, the ice bath was applied again and the reaction mixture was stirred for ˜15 min. The reaction product and excess of hydride were decomposed by dropwise addition of water (3 mL), and the orange-colored reaction mixture became thick and gel-like. After diluted HCl (15 mL; diluted from 4 mL of concentrated HCl) aqueous solution was added dropwise, the solution became clear, and the color changed to light yellow. After the mixture was stirred for several days in N₂, an oily droplet-like phase in the water/THF mixture was observed. After the addition of a few drops of concentrated HCl, the solvent was removed and the residual adhesive oily/greasy droplets were collected using ethanol. The ethanol solution was analyzed with an ESI mass spectrometer. A major peak at m/z 616.22914, corresponding to [M+H]⁺ (m/z 616.2290) of TRI-1(C₃₃H₃₃N₃O₉, monoisotopic mass: 615.2217, theoretical [M+H]⁺: 616.2290) and two medium peaks at m/z 481.16046 and 509.19188 corresponding to [M+H]⁺ (m/z 481.1605) of TRI-2 (C₂₅H₂₄N₂O₈, monoisotopic mass: 480.1533, theoretical [M+H]⁺: 481.1605) and [M+H]⁺ (m/z 509.1918) of TRI-3 (C₂₇H₂₈N₂O₈, monoisotopic mass: 508.1846, theoretical [M+H]⁺: 509.1918) were observed. The mole ratio of TRI-1:TRI-2:TRI-3 is calculated to be 51.4:22.7:25.9.

### Protocol 1: Collection of Dentin Collagen

Collagen is found throughout the body in varying contexts. Therefore, any demonstration of the disclosed collagen crosslinkers and/or crosslinking mixture's beneficial effects works best by selecting a single type of collagen to serve as an example. Dentin collagen was chosen to exemplify the collagen-biostabilizing properties of the herein disclosed crosslinking mixture because the stabilization of dentin is so frequently a concern in dental medicine.

Dentin collagen films were collected and prepared according to the following protocol. Thirty-six non-carious human third molars were collected following a protocol wherein twelve teeth were cut into dentin blocks (6×6×4 mm) after removing enamel layer in a slow speed diamond saw under water cooling. Later, these 12 dentin blocks were processed into 876 dentin films (6-μm thick) with a tungsten carbide knife mounted on microtome. These films were fully demineralized in 10% phosphoric acid and rinsed for 30 minutes in deionized water. Then, the demineralized dentin collagen films were randomly divided into 12 experimental groups (N=73 per group).

### Protocol 2: Exposure of Dentin Films to Crosslinkers

The crosslinking ability and biostability promoted by the herein disclosed collagen crosslinker mixture were compared to other four known crosslinkers: EGCG, QC, GEN, and EDC/NHS. All the crosslinkers were freshly prepared using 0.6 wt % in EtOH. A group using only EtOH as treatment was the control. The crosslinkers were applied at two different treatment times: 60 s and 1 h. After treatment, all dentin collagen films were thoroughly rinsed in EtOH for 30 min (3×10 min) to remove any chemically unreacted compound.

### Example 1: Biodegradation of Dentin Collagen by Exogenous Collagenase

Following the treatment and rinse, the dentin collagen films were dried (48 h under vacuum) and then exposed to 0.1% collagenase (type I, from Clostridium histolyticum, ≥125 U/mg) degradation for 1 h at 37° C. (n=6×10 films per group). The remnant films were dried as previously described and compared the weight percent before and after collagenase digestion for weight loss analysis (WL). The digestion solution was collected and processed to quantify the amount of HYP released. This processing comprised exposure to 5% Ehrlich's reagent, which binds to HYP and produces a colored solution, the color intensity of which is proportional to the concentration of HYP. Colorimetry was performed by measuring the absorbance at 555 nm in a microplate reader, thereby calculating the HYP μg/mg of collagen during digestion. Data were analyzed by two-way ANOVA and Games Howell's post hoc (α=0.05). All data for weight loss are separately shown after 60 seconds of digestion and 1 hour of digestion for each sample.

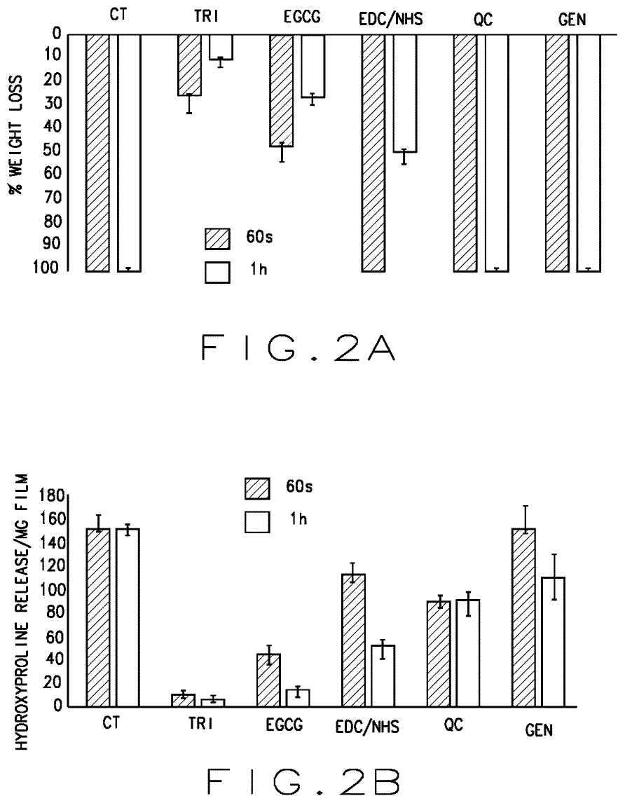

The results are as shown in FIGS. 2A and 2B. FIG. 2A tracks the measured weight % change before and after collagenase digestion. The CT sample, which used only ethanol to protect the dentin collagen, clearly showed very high weight % change, with QC and GEN samples not faring much better. By far the least weight % loss from digestion was observed with samples exposed to the TRI crosslinker mixture. Similarly, FIG. 2B shows that samples exposed to TRI showed the least HYP release during digestion, with competing crosslinkers faring significantly worse in this regard, and the control sample performing worst, as one might expect.

### Example 2: Direct Inactivation of Exogenous Collagenase by TRI

The direct inactivation of collagenase by TRI was also determined using a collagenase assay kit. Assays were performed in 96-well microplates with 0.2 U/ml Clostridium histolyticum collagenase incubated with the treatment solutions for up to 24 h according to the manufacturer's instructions. The well-known collagenase inhibitor 5 mM EDTA was used as a positive control. A group without any treatment was included as a negative control. The fluorescence of kinetic of inactivation was monitored at 1, 2, 4, 16 and 24 h of incubation at 37° C. in triplicate at 490 nm/520 nm. Fluorescence at the different time points was determined by subtracting background fluorescence. The collagenase activity was expressed in arbitrary fluorescence units. Then, the activity was calculated in percentage of inhibition according to the untreated negative control (presumed 100% collagenase activity) after 4 h of incubation. Statistical analysis was performed by one-way ANOVA and Games Howell's post hoc (α=0.05).

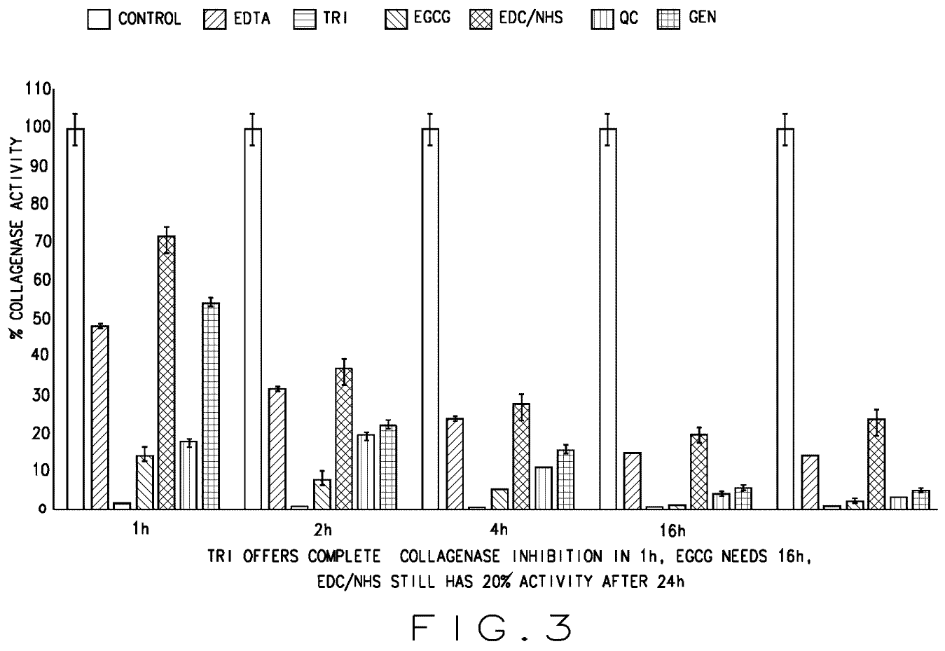

Results are shown in FIG. 3. Although all crosslinkers including EDTA exhibit some collagenase inhibition after 1 hour, TRI samples show the strongest collagenase inhibition by far. Samples exposed to other crosslinking agents did not exhibit comparable collagenase inhibition even after 24 hours of exposure, showing that TRI was not only extremely effective for this purpose, but also inhibited collagenase extremely quickly.

### Example 3: Endogenous MMP Activity by In Situ Zymography

Three dentin collagen films from each group were submitted to analysis of endogenous MMPs within the dentin collagen. A fluorescein-conjugated gelatin of assay kit was prepared immediately before use according to the manufacturer's protocols. Right after each treatment, the collagen films were spread onto microscope glass slides, covered with a drop of the gelatin (3 μL), and then incubated in a humidified chamber protected from light for 24 h at 37° C. Each microscope slide containing the films was covered by coverslips and visualized in a confocal laser scanning microscope in a fluorescence mode (40× objective lens of 0.95 NA) at 488 nm of excitation and 530 nm of emission. Three images obtained from the same z layer were randomly captured for each collagen film. All images (n=9 images for each group) were analyzed and quantified in terms of relative intensities of green fluorescence indicating the activity of the endogenous MMPs. Data were analyzed by two-way ANOVA and Games Howell's post hoc (α=0.05).

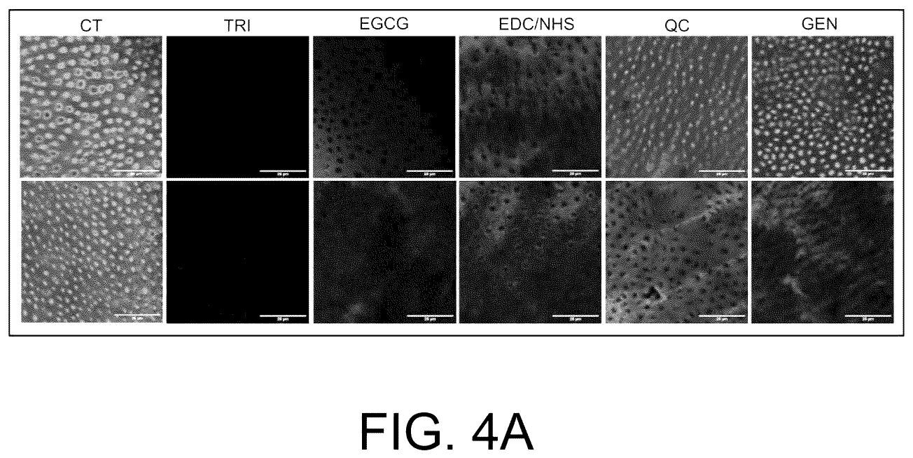

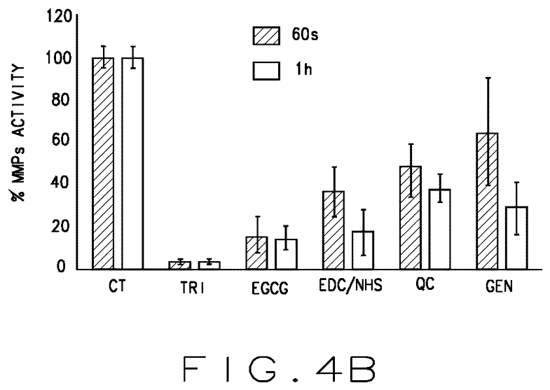

Results are shown in FIGS. 4A and 4B. MMP activity was predictably highest for CT samples, while all samples exposed to crosslinking agents exhibited some inhibition of MMP activity. However, the TRI-treated samples clearly showed the greatest inhibition of MMP.

### Protocol 3: Cell Culture Preparation

Cell cultures were prepared to determine the cytotoxicity of TRI by exposing cells either directly to TRI, or indirectly to substances released from TRI-treated dentin films. NCTC clone 929 fibroblasts were cultured in Eagle's Minimum Essential Medium (EMEM) supplemented with 10% FBS and 1% solution of 1:1 penicillin: streptomycin. The cells were seeded onto 24 and 96-well plates at a density of 2×104 cells/cm² and incubated at 37° C. in 5% CO₂ and 95% relative humidity until the monolayer cells spread over the bottom of the wells.

### Example 4: Effect of Direct Treatment on Cell Proliferation

The direct treatments were conducted on 96-well plates with replacement of original culture medium with culture medium containing different diluted treatment solutions at ratios of 1:1000, 1:2000 and 1:4000 (v/v) and a control group containing only original culture medium. Cell proliferation was assessed with the MTS assay CellTiter 96 AQᵤₑₒᵤₛ One Solution Cell Proliferation Assay following protocol guidelines specified by the manufacturer. Absorbance was measured using a multimode microplate reader at 460/30 nm at 2 h after addition of MTS reagent. Statistical analysis was performed using a two-way ANOVA for each used concentration followed by Tukey's post hoc (α=0.05).

Results are shown in FIG. 5. Even at the highest concentrations, cell cultures treated with TRI show excellent cell proliferation properties as compared to the control sample. GA (glutaraldehyde), on the other hand and as expected, shows high cytotoxicity.

### Example 5: Effect of Released Substances on Cell Proliferation

Twenty-four teeth were sectioned obtaining 24-dentin disks (approximately 6-mm in diameter). The dentin disks were superficially etched using 32% phosphoric acid gel for 15 s on each side, rinsed thoroughly and placed in 100 μL of treatment solutions. After 1 min of treatment, the treated dentin disks were rinsed in sterile distilled water for 30 s. Treated dentin disks were immediately placed on a permeable polycarbonate membrane insert (6.5 mm in diameter, 3.0 μm pore size) above of cultured cells and covered with 100 μL of medium. After incubation, cell viability assay was used to stain live cells with calcein-AM and dead cells with ethidium homodimer. To distinguish overlapped cells in cell counting, Hoechst 33342 was used as a cell-permeant nuclear counterstain, following the guidelines specified by the manufacturer. Cells were imaged using a fluorescent High Content Microscope with 20× magnification. Cell counting was performed using ImageJ (NIH Image J1.8.0). The percentage of cell proliferation and dead cells were statistically compared to 1-day control using one-way ANOVA and Dunnett's test (α=0.05).

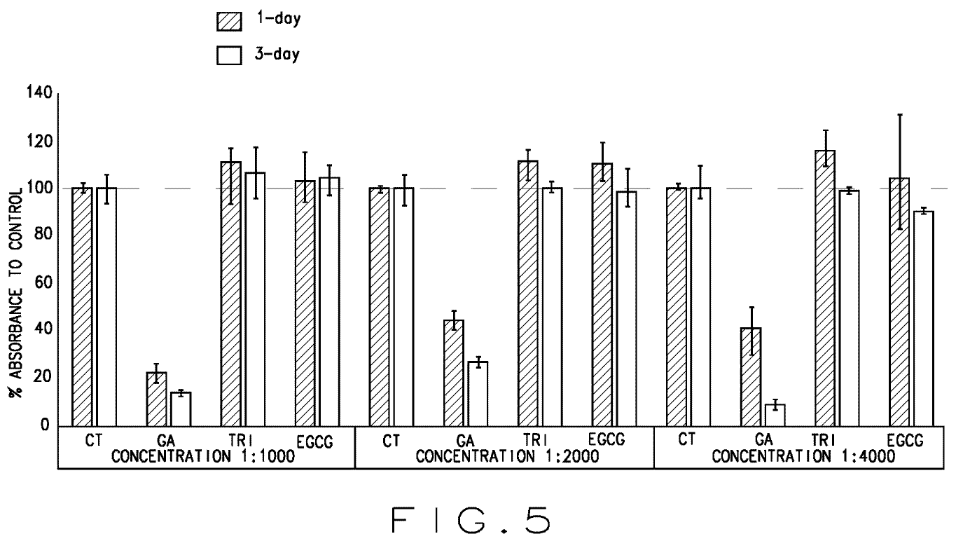

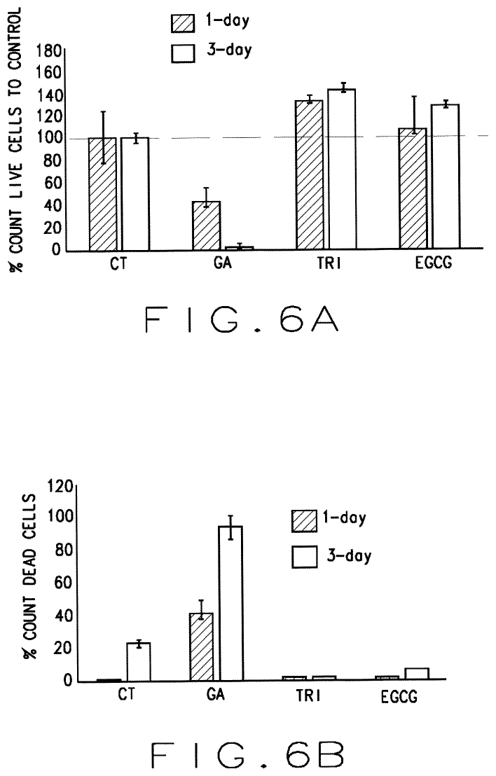

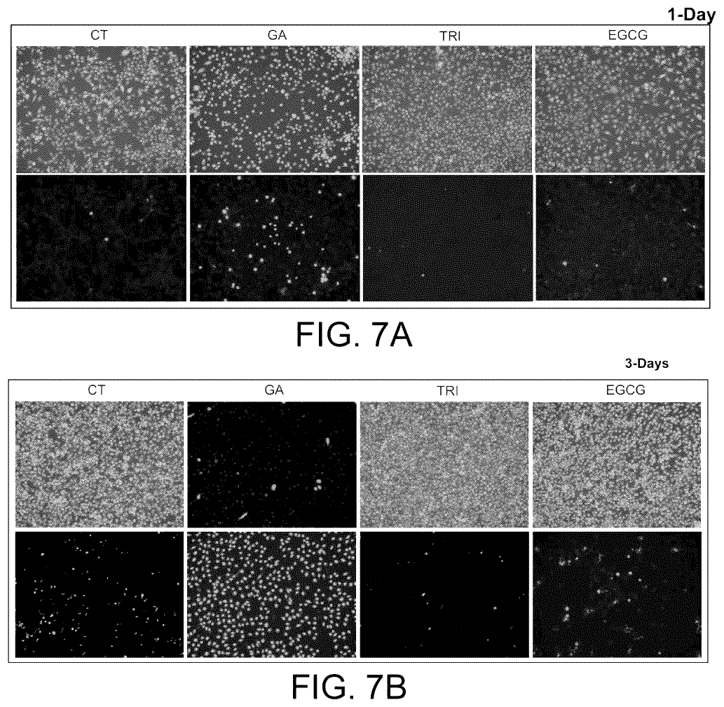

Results from this analysis can be seen in FIGS. 6A-B and 7A-B. When compared against the control, treatment with TRI resulted in a higher number of live L929 cells and a minimum of dead L929 cells detected. GA, by contrast, is cytotoxic in its effects.

### Example 6: Addition of Tri to Dental Adhesive and Effect on Collagen Degradation and Adhesive Degree of Conversion

The inventors recognize that the use of the TRI crosslinkers in various applications may require intermixing with other substances. For example, the use of TRI crosslinkers in stabilizing dentin collagen is likely to be concomitant with the use of a dental adhesive as part of a medical therapy. To date, there is no collagen crosslinker available that offers strong dentin collagen stabilization, a long shelf life attributable to an inherent chemical stability, colorlessness for aesthetic appeal, non-toxicity, and non-interference with dental adhesive polymerization.

Thus, in a preliminary study, we tested the addition of a 0.6% TRI solution mixed directly with a commercial dental adhesive at 0, 5, 10, and 15% (v/v). These correspond to final TRI concentrations of 0, 0.03, 0.06, and 0.09% TRI when mixed with the dental adhesive. Dentin collagen was treated for 60 s with each of these mixtures followed by a thorough rinse in EtOH for 30 minutes.

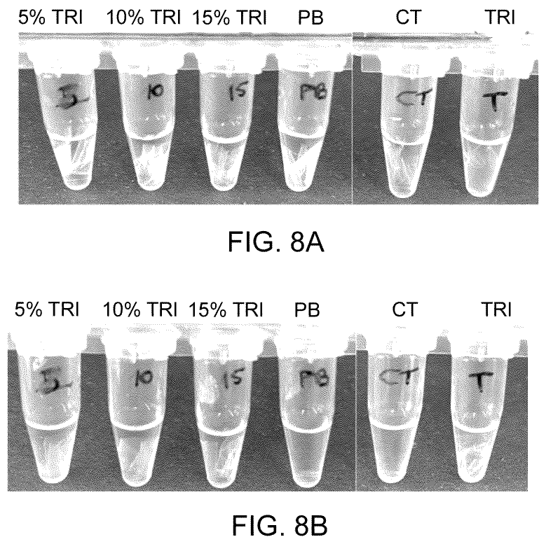

Exposure of these samples to collagenase was tested. Visual results are shown in FIGS. 8A and 8B, which show microcentrifuge tubes of each sample before (FIG. 8A) and after (FIG. 8B) 1 hour of collagenase digestion. The two rightmost samples in each figure are the CT solutions, which did not contain any TRI or adhesive. Visual assessment confirms that in the absence of TRI (0%), dentin collagen was completely digested after 1 hour, whereas each sample containing TRI showed protection of the collagen. Thus, there does not appear to have been a deleterious effect on the TRI's collagenase inhibition from mixing with the dental adhesive.

The inverse was also tested: whether TRI has a deleterious effect on the polymerization ability of the dental adhesive monomers. Fourier transform infrared spectroscopy (FTIR) with a universal Attenuated Total Reflectance (ATR) accessory was used for the photopolymerization test. An LED light curing unit was used for specimen irradiation. Five adhesive samples were tested: Prime&Bond Elect without any cross-linker (PB), PB with 5% and 10% (v/v) TRI and PB with 5% and 10% (v/v) GSE. Adhesive samples were placed on the diamond crystal top plate of the ATR attachment at a thickness of 0.3 mm, covered with a plastic coverslip and light-cured for 10 s. Degree of conversion at 10 min post curing was calculated using the 811/1716 cm⁻¹ band ratio (n=5).

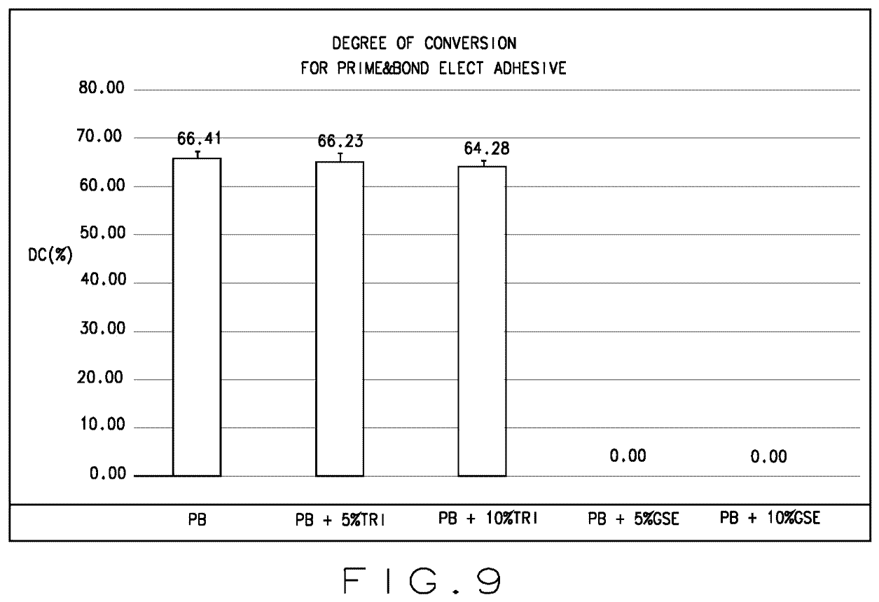

Results can be seen in FIG. 9. According to the results, the PB+5% TRI adhesive showed the same degree of conversion (DC) value (66.23+1.22%) as the PB adhesive (66.41+0.85%), the PB+10% TRI adhesive showed slightly lower DC value (64.28+1.39%) than the PB adhesive, while the PB adhesive containing 5% or 10% GSE showed zero DC value. The results suggest that the TRI crosslinker has minimal interference with the photo polymerization of the PB adhesive at ≤10% concentration, while GSE at 5-10% concentration completely stops the photo polymerization of the PB adhesive.

In view of the above, it will be seen that the several objects and advantages of the present invention have been achieved and other advantageous results have been obtained. The collagen crosslinker mixture of the present disclosure is non-cytotoxic and actually encourages cell proliferation. It furthermore exhibits strong inhibitions of collagenase and MMP, stabilizing collagen against degradation even in low concentrations and when mixed with commercial dental adhesives. Being colorless and fast-acting, providing collagen stability against collagenases after a mere 60 seconds, it is ideal for use with repair and protection of collagen-containing bodily tissues, including those that are visible to the human eye.

As various changes could be made in the above constructions without departing from the scope of the invention, it is intended that all matter contained in the above description or shown in the accompanying drawings shall be interpreted as illustrative and not in a limiting sense.

1. A collagen crosslinker mixture comprising at least one of:
a collagen crosslinker TRI-1 having the structure: 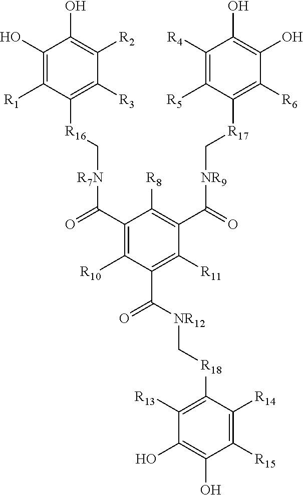
wherein R₁-R₁₅ are each independently selected from the group consisting of hydrogen, halides, hydroxides, amines, linear alkyls, branched alkyls, cyclic alkyls, heteroalkyls, alkenyls, and alkynyls, and further wherein R₁₆-R₁₈ are each independently selected alkyl groups;
a collagen crosslinker TRI-2 having the structure: 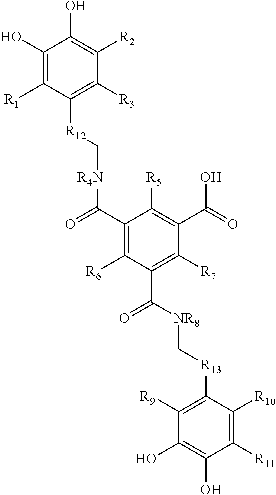
wherein R₁-R₁₁ are each independently selected from the group consisting of hydrogen, halides, hydroxides, amines, linear alkyls, branched alkyls, cyclic alkyls, heteroalkyls, alkenyls, and alkynyls, and further wherein R₁₂ and R₁₃ are independently selected alkyl groups; and,
a collagen crosslinker TRI-3, having the structure: 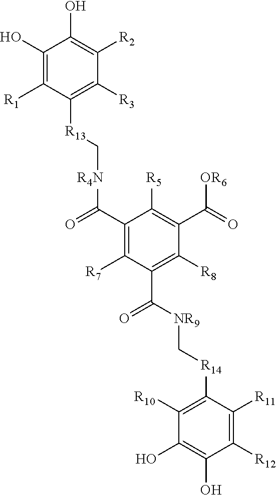
wherein R₁-R₁₂ are each independently selected from the group consisting of hydrogen, halides, hydroxides, amines, linear alkyls, branched alkyls, cyclic alkyls, heteroalkyls, alkenyls, and alkynyls, and further wherein R₁₃ and R₁₄ are independently selected alkyl groups.

2. The mixture of claim 1 wherein every ‘-R’ substituent on TRI-1, TRI-2, and TRI-3 is hydrogen.

3. The mixture of claim 1 wherein the ratio of TRI-1:TRI-2:TRI-3 is approximately 2:1:1.

4. A method for producing a collagen crosslinker mixture, comprising the steps of:
mixing a tricarbonyl benzene compound with a catecholamine to generate a mixture comprising molecules of the forms TRI-1 and TRI-2, with TRI-1 having the structure: 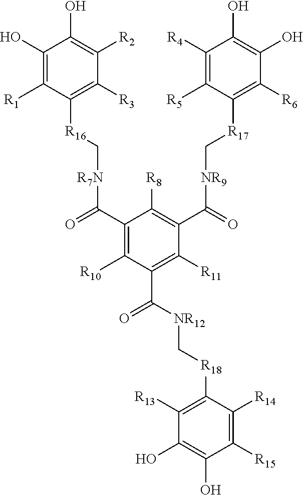
wherein R₁-R₁₅ are each independently selected from the group consisting of hydrogen, halides, hydroxides, amines, linear alkyls, branched alkyls, cyclic alkyls, heteroalkyls, alkenyls, and alkynyl, and further wherein R₁₆-R₁₈ are independently selected alkyl groups;
and with TRI-2 having the structure: 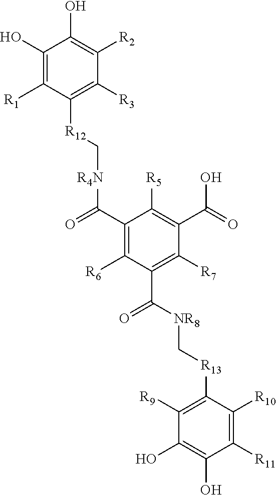
wherein R₁-R₁₁ are each independently selected from the group consisting of hydrogen, halides, hydroxides, amines, linear alkyls, branched alkyls, cyclic alkyls, heteroalkyls, alkenyls, and alkynyls, and further wherein R₁₂ and R₁₃ are independently selected alkyl groups;
reacting the mixture with a reducing agent; and,
adding an alcohol to the mixture to obtain a mixture comprising TRI-1, TRI-2, and TRI-3, with TRI-3 having the structure: 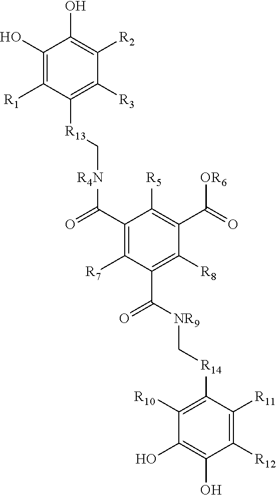
wherein R₁-R₁₂ are each independently selected from the group consisting of hydrogen, halides, hydroxides, amines, linear alkyls, branched alkyls, cyclic alkyls, heteroalkyls, alkenyls, and alkynyls, and further wherein R₁₃ and R₁₄ are independently selected alkyl groups.

5. The method of claim 4 wherein said tricarbonyl benzene is trimesoyl chloride.

6. The method of claim 4 wherein said catecholamine is dopamine.

7. The method of claim 4 wherein said reducing agent is LiAlH₄.

8. The method of claim 4 wherein the alcohol is ethanol.

9. A dentin treatment mixture, said mixture comprising:
a dental adhesive; and
a collagen crosslinker mixture comprising at least one of:
a collagen crosslinker TRI-1 having the structure: 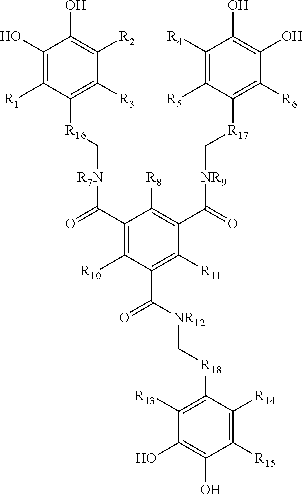
wherein R₁-R₁₅ are each independently selected from the group consisting of hydrogen, halides, hydroxides, amines, linear alkyls, branched alkyls, cyclic alkyls, heteroalkyls, alkenyls, and alkynyls, and further wherein R₁₆-R₁₈ are independently selected alkyl groups;
a collagen crosslinker TRI-2 having the structure 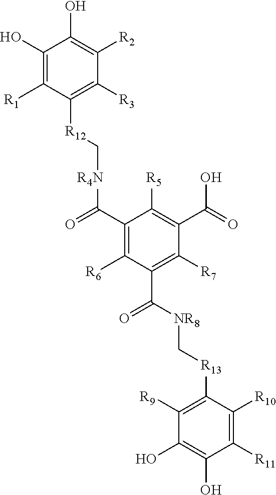
wherein R₁-R₁₁ are each independently selected from the group consisting of hydrogen, halides, hydroxides, amines, linear alkyls, branched alkyls, cyclic alkyls, heteroalkyls, alkenyls, and alkynyls, and further wherein R₁₂ and R₁₃ are independently selected alkyl groups; and,
a collagen crosslinker TRI-3, having the structure: 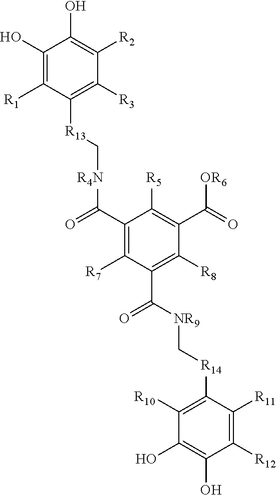
wherein R₁-R₁₂ are each independently selected from the group consisting of hydrogen, halides, hydroxides, amines, linear alkyls, branched alkyls, cyclic alkyls, heteroalkyls, alkenyls, and alkynyls, and further wherein R₁₃ and R₁₄ are independently selected alkyl groups
